(19) United States
(12) Patent Application Publication
(10) Pub. No.: US 2026/0284318 A1
(43) Pub. Date: Sep. 24, 2026

(54) MIXING SYRINGE ASSEMBLIES AND METHODS

(71) Applicant: CLEARSTREAM TECHNOLOGIES LIMITED, Enniscorthy, County Wexford (IE)

(72) Inventor: Matthew Cullen, Wicklow Town (IE)

(73) Assignee: CLEARSTREAM TECHNOLOGIES LIMITED, Enniscorthy, County Wexford (IE)

(21) Appl. No.: 19/473,397

(22) PCT Filed: Apr. 26, 2024
(86) PCT No.: PCT/US2024/026493

§ 371 Date: Oct. 7, 2025

Related U.S. Application Data
(60) Provisional application No. 63/498,656, filed on Apr. 27, 2023.

(51) Int. Cl. A61M 5/28; A61M 5/19; A61M 5/31

(52) U.S. Cl. CPC A61M 5/284; A61M 5/19; A61M 5/286; A61M 2005/3128; A61M 2207/00

(57) ABSTRACT

A mixing syringe assembly includes a barrel body and a sealing device. The barrel body includes a track defining a first longitudinal path, a second longitudinal path, and a rotational path. The first longitudinal path and the second longitudinal path are rotationally offset from one another by the rotational path. The sealing device is positioned in a dividing position between within the rotational path, the sealing device dividing the barrel body into a first constituent material section and a second constituent material section. The sealing device is rotatable within the rotational path allowing the sealing device to enter the second longitudinal path and be advanced along the second longitudinal path. The sealing device is operable to adjust from a sealing configuration to a non-sealing configuration to allow for first constituent material from the first constituent material section to move into the second constituent material section holding second constituent material.

### CROSS-REFERENCE TO RELATED APPLICATIONS

The present application claims the benefit of U.S. Provisional Application No. 63/498,656, entitled “MIXING SYRINGE ASSEMBLIES AND METHODS OF MIXING CONSTITUENT MATERIALS,” filed April, 27, 2024, the entirety of which is incorporated by reference herein.

### BACKGROUND

### Field

The present disclosure generally relates to mixing syringe assemblies and methods and, more particularly, mixing syringe assemblies for delivering radiation spacer material and associated methods.

### Technical Background

Prostate cancer is the most common non-skin cancer diagnosed in men. Radiation therapy is an excellent treatment option for prostate cancer. However, radiation exposure can cause unintended side effects in adjacent non-targeted tissue. A radiation spacer, such as a radio protective spacer) can be implanted to avoid collateral radiation and minimize injury to nearby tissues by providing a space between the target tissue and non-targeted tissues at risk.

Conventional radiation spacers may include gelling materials, which are delivered as a liquid and allowed to cure. However, constituent components of the gel may begin to gel on contact with one another, which may make delivery difficult. Moreover, constituent components may need to remain separate until time of use, otherwise they may degrade over time due to limited stability.

### SUMMARY

Embodiments of the present disclosure are directed to various mixing syringe assemblies, which may maintain separation of constituent materials until time for use of the mixed constituent materials.

In one embodiment, a mixing syringe assembly includes a barrel body and a sealing device. The barrel body includes a track defining a first longitudinal path, a second longitudinal path, and a rotational path wherein the first longitudinal path and the second longitudinal path are rotationally offset from one another by the rotational path. The sealing device is configured to be positioned in a dividing position within the rotational path dividing the barrel body into a first constituent material section and a second constituent material section, wherein the sealing device is rotatable within the rotational path such that rotation of the sealing device within the rotational path allows the sealing device to enter the second longitudinal path and be advanced along the second longitudinal path, and wherein the sealing device is operable to adjust from a sealing configuration to a non-sealing configuration to allow for a first constituent material from the first constituent material section to move into the second constituent material section holding a second constituent material.

In another embodiment, a mixing syringe assembly includes a first mixing syringe subassembly and a second mixing syringe subassembly coupled to the first mixing syringe subassembly. Each mixing syringe subassembly includes a barrel body and a sealing device. The barrel body includes a track defining a first longitudinal path, a second longitudinal path, and a rotational path wherein the first longitudinal path and the second longitudinal path are rotationally offset from one another by the rotational path. The sealing device is configured to be positioned within a dividing position within the rotational path, the sealing device dividing the barrel body into a first constituent material section and a second constituent material section, wherein the sealing device is rotatable within the rotational path such that rotation of the sealing device within the rotational path allows the sealing device to enter the second longitudinal path and be advanced along the second longitudinal path, and wherein the sealing device is operable to adjust from a sealing configuration to a non-sealing configuration to allow for a first constituent material from the first constituent material section to move into the second constituent material section holding a second constituent material.

In yet another embodiment, a method of assembling a mixing syringe assembly includes inserting a first constituent material into a barrel body, wherein the barrel body comprises a track defining a first longitudinal path, a second longitudinal path, and a rotational path wherein the first longitudinal path and the second longitudinal path are rotationally offset from one another by the rotational path, and inserting a sealing device into the barrel body such that the sealing device is positioned within the rotational path and divides the barrel body into a first constituent material section and a second constituent material section. The sealing device is rotatable within the rotational path such that rotation of the sealing device within the rotational path allows the sealing device to enter the second longitudinal path and be advanced along the second longitudinal path. The sealing device is operable to adjust from a sealing configuration to a non-sealing configuration to allow for the first constituent material from the first constituent material section to move into the second constituent material section holding a second constituent material.

Additional features and advantages of the aspects described herein will be set forth in the detailed description, which follows, and in part will be readily apparent to those skilled in the art from that description or recognized by practicing the aspects described herein, including the detailed description, which follows, the claims, as well as the appended drawings.

It is to be understood that both the foregoing general description and the following detailed description describe various aspects and are intended to provide an overview or framework for understanding the nature and character of the claimed subject matter. The accompanying drawings are included to provide a further understanding of the various aspects, and are incorporated into and constitute a part of this specification. The drawings illustrate the various aspects described herein, and together with the description serve to explain the principles and operations of the claimed subject matter.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1 schematically depicts an embodiment of a syringe assembly for mixing constituent materials, according to one or more embodiments shown and described herein;

FIG. 2 schematically depicts a dual syringe assembly for mixing constituent materials, according to one or more embodiments shown and described herein;

FIG. 3A schematically depicts a sealing device separating a barrel into a first constituent material section and a second constituent material section, according to one or more embodiments shown and described herein;

FIG. 3B schematically depicts a first constituent material moving from the first constituent material section into the second constituent material section through the sealing device of FIG. 3A, according to one or more embodiments shown and described herein;

FIG. 3C schematically depicts advancing a plunger and the sealing device distally in the barrel of FIG. 3B, according to one or more embodiments shown and described herein;

FIG. 4 depicts a perspective view of a dual barrel, according to one or more embodiments shown and described herein;

FIG. 5 depicts a flow chart illustrating a method of assembling a mixing syringe assembly, according to one or more embodiments shown and described herein; and FIG. 6 depicts a flow chart illustrating a method of using a mixing syringe assembly, according to one or more embodiments shown and described herein.

### DETAILED DESCRIPTION

The present disclosure is generally directed to mixing syringe assemblies for mixing at least two constituent materials. Particularly, the present application is directed to mixing syringe assemblies configured for delivering a radiation spacer. For example, radiation spacers may be formed of one or more hydrogel materials, which may be delivered to a desired location, such as within a balloon or on its own and cured in place to block or substantial block radiation, which may be unintentionally directed toward healthy tissue instead of targeted/diseased tissue. During mixing of the hydrogel for forming a radiation spacer, multiple materials may need to be combined just prior to and/or at delivery of the radiation spacer into a target location within the body. To maintain quality of material, powder components may need to be kept dry or un-constituted prior to application. However, during use, powder components may need to be hydrated or mixed with a diluent. It may be difficult to determine proper mixing volumes, maintain sterility, and prevent unwanted early mixing using traditional methods. However, embodiments of the present disclosure may provide constituent materials in prepackaged, pre-measured, ready-to use syringe assemblies, which, as will be described in greater detail herein, provide improved mixing, maintains sterility, and/or improves delivery.

Particular embodiments of a mixing syringe assembly include a barrel body, a plunger and a sealing device. The sealing device is operable to divide a barrel body into a first constituent material section and a second constituent material section, thereby maintaining separation of a first constituent material in the first constituent material section and a second constituent material within the second constituent material section until the seal device is actuated from a closed configuration to an open configuration. In embodiments of the present disclosure a barrel or barrel body includes a track and a sealing device which separates the barrel body into a first constituent material section and a second constituent material section. The sealing device may maintain fluid constituent materials from powder constituent materials until desired combination. Once delivery is desired, the sealing device is movable along the track to aid in delivery of the combined materials. These and additional embodiments and benefits will be described in greater detail below.

Directional terms as used herein—for example up, down, right, left, front, back, top, bottom—are made only with reference to the figures as drawn and are not intended to imply absolute orientation unless otherwise specified.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any device or assembly claim does not actually recite an order or orientation to individual components, or it is not otherwise specifically stated in the claims or description that the steps are to be limited to a specific order, or that a specific order or orientation to components of an device or assembly is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation; and the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

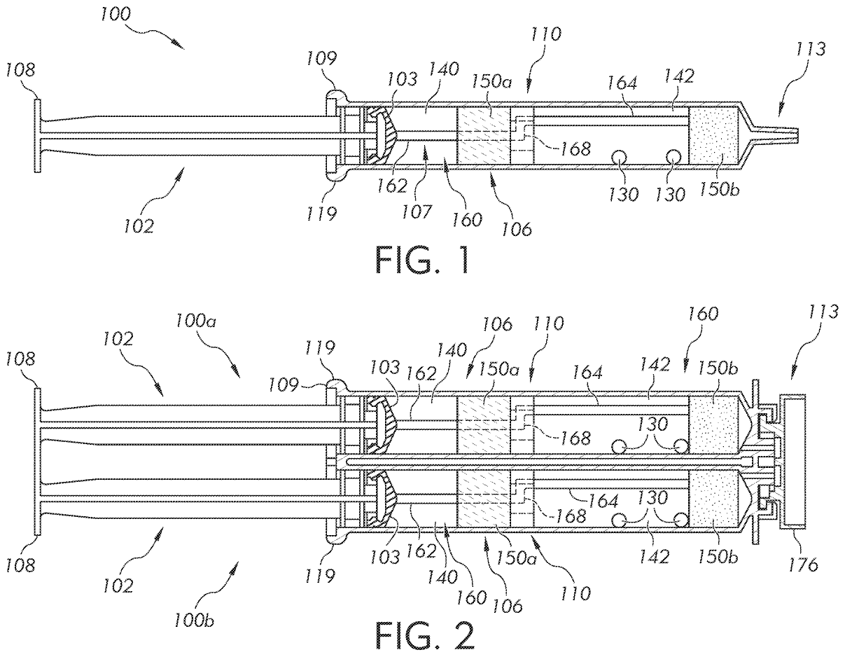

Turning now to the drawings, FIG. 1 depicts an illustrative mixing syringe assembly 100. The mixing syringe assembly 100 may be used for combining two or more constituent materials as will be described in greater detail herein. In particular, the mixing syringe assembly 100 may be used to mix or combine two or more constituent components of a hydrogel for use as a radiation spacer. The mixing syringe assembly 100, as depicted, generally includes a plunger 102, a barrel body 106, and a sealing device 110. In some embodiments, the mixing syringe assembly 100 further includes one or more mixing masses 130. It is noted that a mixing syringe assembly 100 may include a greater or fewer number of components without departing from the scope of the present disclosure.

The plunger 102 may be slidably disposed in a lumen 107 of the barrel body 106 and may form a fluidic seal with the barrel body 106 via a seal 103 at a distal end of the plunger 102. For example, the seal 103 may be a rubber or similar material conformable material for forming a fluid-tight seal with the barrel body 106. At a proximal end of the plunger 102 may be a pusher flange 108 for a user to engage for advancing the plunger 102 along the barrel body 106.

The barrel body 106 may be generally hollow and have plunger-receiving end 109 at a proximal end for receiving the plunger 102 and a fluid delivery opening 113 at a distal end, which may be coupled to a material delivery cannula (not shown, which may include a manifold, delivery needle, or the like). In embodiments, the fluid delivery opening 113 may be axially positioned along a centerline of the barrel body 106 and/or plunger 102. However, in other embodiments, the fluid delivery opening 113 may be offset from a centerline of the barrel body 106 and/or plunger 102. Such offset may be useful in preventing the one or more mixing masses 130 from blocking the fluid delivery opening 113.

The sealing device 110 initially separates the barrel body 106 into a first constituent material section 140 and a second constituent material section 142 when in a dividing position. The first constituent material section 140 and the second constituent material section 142 may initially take up the same volume or substantially same volume as depicted. However, in some embodiments, initial volumes may differ from one another. As will be described in greater detail, a first constituent material 150a may be positioned initially within the first constituent material section 140 and a second constituent material 150b, different from the first constituent material 150a, may be positioned within the second constituent material section 142. As depicted in the illustrated embodiment, the first constituent material section 140 may be positioned proximal to the second constituent material section 142.

The first constituent material 150a may be a liquid for hydrating the second constituent material 150b, which may be a powder or particulate. For example, the first constituent material 150a may be saline, water, deionized water, or the like. As noted above, the second constituent material 150b may be a powder or particulate material such as but not limited to albumin, polyethylenimine (PEI), an amine containing polyethylene glycol (PEG) or protein, an N-hydroxysuccinimide (NHS) ester component such as PEG-(SS)2, PEG-(SS)4, PEG-(SS)8, PEG-(SG)4, PEG-(SG)8, and/or the like. In some aspects, molecular weights of the PEG components may range from about 2,000 to about 100,000. The powder or particular material may be biodegradable and/or bioabsorbable. As used herein, “biodegradable” and/or “bioabsorbable” refers to a compound that can be absorbed by the surrounding or local tissue of a subject and/or degraded and absorbed by the tissue of the subject.

The powder or particulate material can be composed of various crosslinking substances of varying amounts, designed to allow the hydrogel to last a specific amount of time in situ before degrading. In aspects, the hydrogel components may be selected based on a degradation time that corresponds to the length of anticipated radiation therapy. In aspects the length of anticipated radiation therapy, and thus the targeted time for hydrogel degradation is up to 18 months, for example from the range of about 0 months to about 18 months, including about 1 month, 2 months, 3 months, 4 months, 5 months, 6 months, 7 months, 8 months, 9, months, 10 months, 11 months, 12 months, 13 months, 14 months, 15 months, 16 months, 17 months, and 18 months. It should be understood that the time is merely a rough guide generally used to target appropriate formulation of the hydrogel.

The sealing device 110 may take on several different forms. In general though, the various sealing devices described herein may move or are otherwise be modified to selectively provide a flow path between the first constituent material section 140 and the second constituent material section 142 to allow for the first constituent material 150a from the first constituent material section 140 to move into the second constituent material section 142 to allow for mixing of the first constituent material 150a with the second constituent material 150b.

The one or more mixing masses 130 may initially be positioned within either the first constituent material section 140 or the second constituent material section 142. It is noted that in embodiments, placing the one or more mixing masses 130 within the second constituent material section 142 may be beneficial in that a flow path large enough to allow the one or more mixing masses 130 through need not be created to move the one or more mixing masses 130 from the first constituent material section 140 to the second constituent material section 142. The one or more mixing masses 130 may be any suitable material to assist in mixing materials such as stainless steel, delron, plastic polycarbonate, composite, aluminum, low carbon steel, chemical-resistant PTFE, composite, titanium, moisture resistant polyethylene, magnetic material, etc. In one or more mixing masses 130, may be any shape such as spherical, cylindrical, cubic, ellipsoid, or any other regular or irregular shape. The one or more mixing masses 130 may include any number of masses such as two or more, three or more, four or more etc. The one or more mixing masses 130 may be identical to one another or different from one another. The one or more mixing masses 130 may have various sizes such as up to 7 mm in diameter, such as up to 6 mm in diameter, such as up to 5 mm in diameter, such as up to 4 mm in diameter, such as up to 3 mm in diameter, such as 7 mm or less in diameter. In various embodiments, the one or more mixing masses 130 may have designs cut or etched into its surface, which may enhance mixing.

Referring now to FIG. 2, in various embodiments, a mixing syringe assembly 100 may include one or more mixing syringe subassemblies 100a, 100b, such as to provide a dual barrel syringe. For example, FIG. 2 illustrates a dual-mixing syringe assembly 100 including a first mixing syringe subassembly 100a and a second mixing syringe subassembly 100b. Each subassembly 100a, 100b is substantially identical to the mixing syringe assembly 100 described with respect to FIG. 1. In particular, each subassembly 100a may include a plunger 102, a barrel body 106, and a sealing device 110. In some embodiments, the subassemblies 100a, 100b further include one or more mixing masses 130. Accordingly, the above description is applicable to the present embodiments and will not be repeated. In some embodiments, the barrel bodies 106 may be rigidly coupled to one another. For example, the adjacent barrel bodies 106 may be integrally formed or coupled to another via a bracket, welding, adhesive or the like. In some embodiments, a manifold 176 may couple the fluid delivery openings 113 to respective delivery needles or cannulas (not shown) for delivery of fluid. It is noted that in various embodiments, it may be desirable to keep mixed constituent materials from the first subassembly 100a and the second subassembly 100b separate until the point of delivery at a target delivery location, such as within a subject. Accordingly, dual needle assemblies and/or coaxial needle assemblies such as described in International Patent Application No. PCT/US2021/023171, entitled “Multi-Component Sealant Delivery Systems Incorporating Quarter Turn Connectors,” filed Mar. 19, 2021, the entirety of which is hereby incorporated by reference. For example, while the first mixing syringe subassembly 100a may include the first constituent material 150a and the second constituent material 150b, the second mixing syringe subassembly 100b may hold a different first constituent material 150a and/or a different second constituent material 150b. For example, a second constituent material 150b of the first mixing syringe subassembly 100a may hold Peg 8, having 20 kmw, while the second constituent material 150b of the second mixing syringe subassembly 100b may hold Peg 8 having 15 kmw. The first constituent materials 150a may also be the same or different from one another and may include, but are not limited to, water saline, deionized water, or like. In embodiments, the resulting mixtures or solutions within each of the first and second subassemblies 100a, 100b, may gel or solidify on contact with one another. Accordingly, it may be desirable to only allow contact at the moment of delivery, such as at the end of a dual-lumen 107 cannula, such as disclosed in International Patent Application No. PCT/US2021/023171, entitled “Multi-Component Sealant Delivery Systems Incorporating Quarter Turn Connectors,” filed Mar. 19, 2021, the entirety of which is hereby incorporated by reference.

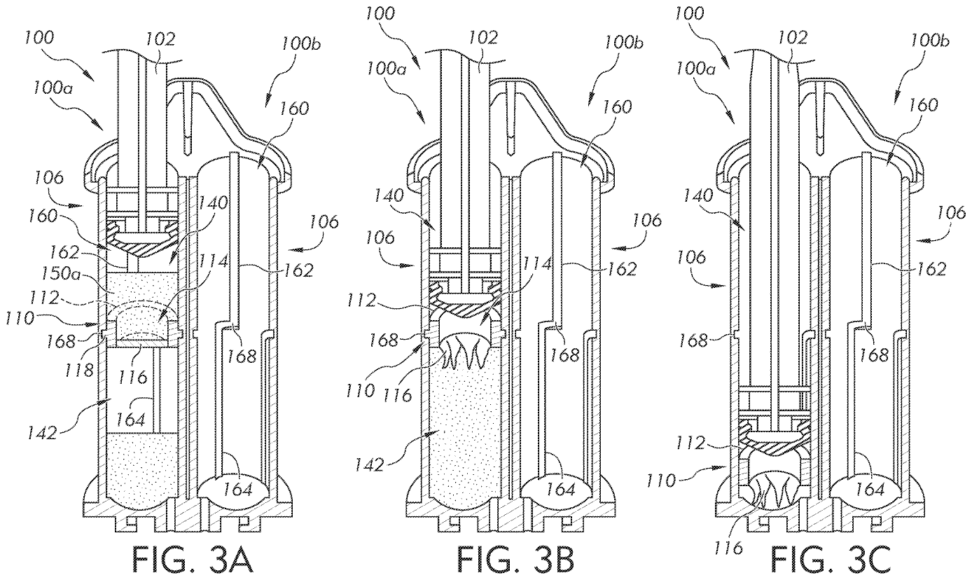

FIG. 3A-3B illustrate use of the mixing syringe subassembly 100 in a dual barrel assembly 100. However, in the depiction the barrel body 106 of the second subassembly 100b is empty to provide better depiction of the internal structure of each barrel body 106. Such internal structure would also be present within the single barrel design. Furthermore, use/operation of both sides or of a single barrel design would be the same. In particular, each barrel body 106 includes a track 160 defining a first longitudinal path 162, a second longitudinal path 164, and a rotational path 168. The first longitudinal path 162 and the second longitudinal path 164 may extend in parallel with an axial direction of each barrel body 106 or plunger 102. However, the first longitudinal path 162 and the second longitudinal path 164 are rotationally offset from one another by the rotational path 168. For example, the first longitudinal path 162 and the second longitudinal path 164 may be rotationally offset from one another by between 1 degree and 90 degrees, such as between about 5 degrees and 45 degrees, such as between 5 degrees and 25 degrees, etc. The first longitudinal path 162 and the second longitudinal path 164 are coupled to one another via the rotational path 168 to provide a continuous path along the barrel body 106. The rotational path 168 may be extend perpendicular to the axial direction of the barrel body 106 or plunger 102. In some embodiments, the rotational path 168 need not be perpendicular to the axial direction of the barrel body 106 or plunger 102 but may be angled at some oblique angle relative to the axial direction of the barrel body 106 or plunger 102. The first longitudinal path 162, the second longitudinal path 164, and the rotational path 168 may be channels recessed into the interior wall of each barrel body 106. In other embodiments, it is contemplated that the first longitudinal path 162, the second longitudinal path 164, and the rotational path 168 may protrude from the interior wall of each barrel body 106.

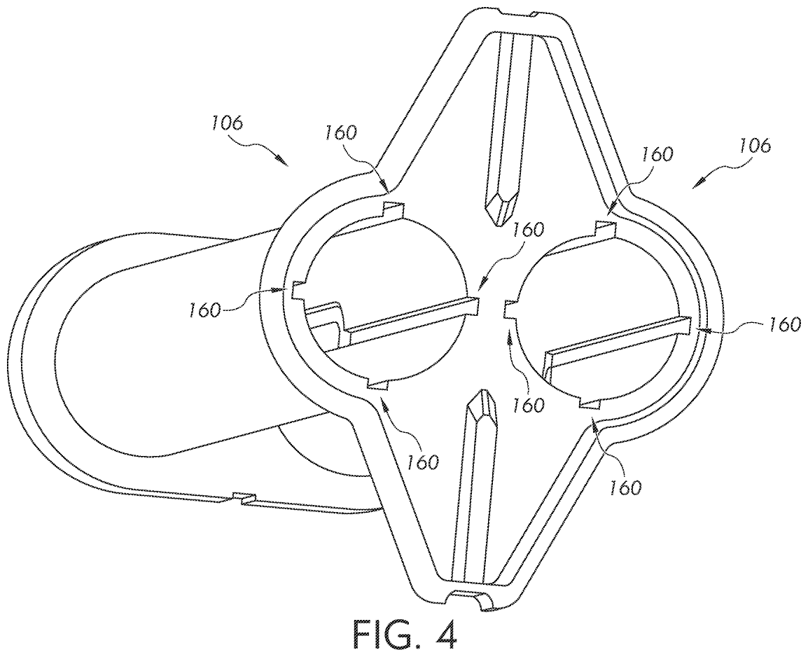

As best depicted in FIG. 4, each barrel body 106 may include a plurality tracks 160. For example, each barrel body 106 may include two or more tracks 160, such as four tracks 160. The plurality of tracks 160 may be equally spaced from one another or unequally spaced. Each of the tracks 160 include the above described first longitudinal path 162, second longitudinal path 164, and rotational path 168. It is noted that in some embodiments, the rotational path 168 may go in an opposite direction in one barrel body 106 versus the other barrel body 106, which may lead to opposite rotation directions for traversing the tracks 160.

Referring again to FIGS. 3A-3C, in embodiments, the sealing device 110 may include any number of sealing devices such as a check valve, a one-way valve, a film valve, or the like. In the depicted embodiment of FIGS. 3A-3C, the sealing device 110 is depicted as a film valve having a valve body 112 defining a flow path 114 therethrough and a burstable film 116 coupled to the valve body 112 (such as via welding, adhesive, or the like) and blocking the flow path 114. The burstable film 116 may be plastic, foil, or the like, and may be configured to burst under pressure, such as supplied by the plunger 102 advancing toward the sealing device 110. In embodiments, the sealing device 110 may engage the track 160 so as to be able to travel along and remain in alignment with the track 160. For example, the sealing device 110 may include a projection 118 which sit within the each track 160, so as to allow the sealing device 110 to be guided by the 160 along the length of the barrel body 106. Accordingly, the sealing device 110 may include a corresponding number to projections 118 and tracks 160. The projections 118 may be sized to fit within the track 160 so as to be able to travel along the longitudinal paths 162, 164 and the rotational path 168. The valve body 112 may include any number of conformable materials or the like to prevent leakage around the valve body 112, such as within the track 160.

In an initial dividing position, the sealing device 110 may be inserted, such as, halfway down each barrel body 106, to the rotational path 168 within the track 160, to divide the barrel body 106 into the first constituent material section 140 and the second constituent material section 142. For example, each portion may be 5 ml in volume, though other sizes are contemplated and possible. In embodiments, the first constituent material section 140 may hold a diluent as described above (e.g., water, saline, etc.) and the second constituent material section 142 may hold a powder as described above (e.g., PEG, Albumin, or the like).

Referring to FIG. 3B, the plunger 102 may be pushed down through the barrel body 106 until it makes contact with the sealing device 110 (e.g., the valve body 112). During pushing of the plunger 102, the diluent is forced through the sealing device 110 into the second constituent material section 142, as illustrated in FIG. 3B. For example, the force or pressure generated by advancing the plunger 102 may cause the sealing device 110 to actuate to an open configuration (e.g., a valve may open or the film may burst). The diluent is then able to be mixed with the powder in the second constituent material section 142. The user may then shake the syringe assembly 100, allowing the powder (second constituent material 150b) to dissolve into the diluent. The user may press downward on the plunger(s) 102 to cause the plunger to engage with the sealing device 110, such as the valve body 112. The plunger 102 may be engageable with the sealing device 110 such that rotation of the plunger rotates the sealing device within the rotational path. For example, the plunger 102 may frictionally engage with the sealing device 110 or interlock with the sealing device 110. Once engaged, a user may apply torque to the plunger 102. The torque will rotate the sealing device 110 through the rotational path 168, which allows the projections 118 to enter the second longitudinal path 164. By traversing the rotational path 168, the sealing device 110 may then be pushed downwards to the distal end of the barrel body 106, as illustrated in FIG. 3C. That is, once the sealing device 110 is within this second longitudinal path 164, the user may then apply downward force with no torque to push the plunger 102 and the sealing device 110 to the distal end of the barrel(s) 106, ultimately delivering the gel through the distal end of the assembly 100.

It is noted that while only movement is illustrated through one side of the barrel body 106, the same process may take place in the other side for simultaneous mixing and delivery.

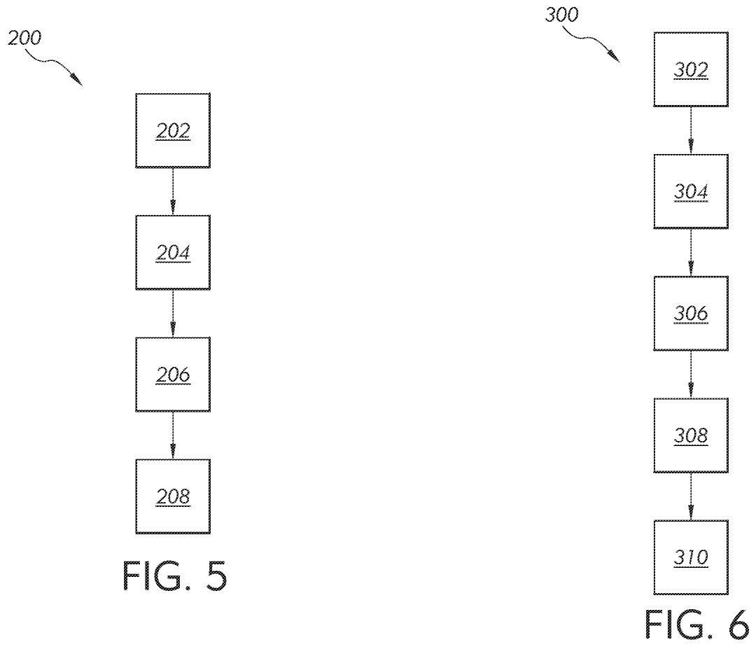

FIG. 5 generally depicts a flow chart illustrating a method 200 of assembling the mixing syringe assembly 100 as described above. The method 200 can include a greater or fewer number of steps then depicted and steps may be performed in any order. The method 200 may include at block 202 inserting the first constituent material 150a into the barrel body 106. At block 204, the method 200 includes inserting the sealing device 110 into the barrel body 106 such that the barrel body 106 is engaged within the rotational path 168 and divides the barrel body 106 into the first constituent material section 140 and the second constituent material section 142. Such step may include inserting the sealing device 110 into the barrel body 106 by engaging the sealing device 110 to the first longitudinal path 162 and advancing the sealing device 110 along the first longitudinal path 162 to the rotational path 168. At block 206, the method 200 may include inserting the second constituent material 150b into the second constituent material section 142. It is noted that a user may first place the second constituent material 150b prior to inserting the sealing device 110 and inserting the first constituent material 150a. The method 200 may further include positioning the plunger with the first constituent material section 140. Accordingly, the mixing syringe assembly 100 may be ready to use, having pre-measured constituent materials therein, which remain isolated from one another via the sealing device.

FIG. 6 generally depicts a flow chart illustrating a method 300 of using the mixing syringe assembly 100 above (either a dual barrel or single barrel type). The method may be best understood in conjunction with FIGS. 3A-3C described above. The method 300 may include at block 302 advancing the plunger 102 toward the sealing device 110 to adjust the sealing device 110 to a non-sealing configuration (e.g., open the valve, burst the film, etc.). In the non-sealing configuration, the method 300 at block 304 includes further advancing the plunger 102 toward the sealing device 110 to transfer the first constituent material 150a into the second constituent material section 142. After, the material first constituent material 150a is delivered into the second constituent material section 142, the method 300 at block 306 includes engaging the plunger 102 with the sealing device 110. Accordingly, when engaged with the sealing device 110 the first constituent material 150a may be fully delivered into the second constituent material section 142. At block 308, the method 300 includes twisting the plunger 102 thereby causing the sealing device 110 to rotate within the rotational path 168. For example, the plunger 102 may interlock or frictionally engage with the sealing device 110 (such as the valve body 112), such that rotation of the plunger 102 causes rotation of the sealing device 110 along the rotational path 168. At block 310, the method 300 further includes advancing the plunger 102 and the sealing device 110 along the second longitudinal path 164. Accordingly, the combined first constituent material 150a and second constituent material 150b may be delivered out of the mixing syringe assembly 100. In embodiments, prior to delivery, the mixing syringe assembly 100 may be agitated to ensure complete mixing of the first constituent material 150a and the second constituent material 150b.

In embodiments including burstable films, rupturing the films may be accomplished via manual actuation or may be assisted with a jig or cradle and pressing actuator, (e.g. a linear actuator mechanically engaged with the plunger 102 and/or the barrel body 106).

Embodiments may be further described with respect to the following numbered clauses:
1. A mixing syringe assembly comprising: a barrel body comprising a track defining a first longitudinal path, a second longitudinal path, and a rotational path wherein the first longitudinal path and the second longitudinal path are rotationally offset from one another by the rotational path; and a sealing device configured to be positioned in a dividing position between within the rotational path, the sealing device dividing the barrel body into a first constituent material section and a second constituent material section, wherein the sealing device is rotatable within the rotational path such that rotation of the sealing device within the rotational path allows the sealing device to enter the second longitudinal path and be advanced along the second longitudinal path, and wherein the sealing device is operable to adjust from a sealing configuration to a non-sealing configuration to allow for a first constituent material from the first constituent material section to move into the second constituent material section holding a second constituent material.
2. The mixing syringe assembly of clause 1, further comprising: a plunger slidably positioned within the barrel body.
3. The mixing syringe assembly of clause 2, wherein the plunger is engageable with the sealing device such that rotation of the plunger rotates the sealing device within the rotational path.
4. The mixing syringe assembly of any preceding clause, wherein the track is recessed within a wall of the barrel body.
5. The mixing syringe assembly of any preceding clause, wherein the sealing device comprises a projection which extends into the track.
6. The mixing syringe assembly of any preceding clause, wherein the sealing device comprises a one-way valve.
7. The mixing syringe assembly of any preceding clause, wherein the sealing device comprises a burstable film.
8. A mixing syringe assembly for mixing multiple constituent components, the mixing syringe assembly comprising: a first mixing syringe subassembly and a second mixing syringe subassembly coupled to the first mixing syringe subassembly, each mixing syringe subassembly comprising: a barrel body comprising a track defining a first longitudinal path, a second longitudinal path, and a rotational path wherein the first longitudinal path and the second longitudinal path are rotationally offset from one another by the rotational path; and a sealing device configured to be positioned within a dividing position between within the rotational path, the sealing device dividing the barrel body into a first constituent material section and a second constituent material section, wherein the sealing device is rotatable within the rotational path such that rotation of the sealing device within the rotational path allows the sealing device to enter the second longitudinal path and be advanced along the second longitudinal path, and wherein the sealing device is operable to adjust from a sealing configuration to a non-sealing configuration to allow for a first constituent material from the first constituent material section to move into the second constituent material section holding a second constituent material.
9. The mixing syringe assembly of clause 8, wherein the barrel body of the first mixing syringe subassembly is coupled to the barrel body of the second mixing syringe subassembly.
10. The mixing syringe assembly of any preceding clause, wherein the first mixing syringe subassembly and the second mixing syringe subassembly further comprise a plunger slidably positioned within the barrel body.
11. The mixing syringe assembly of clause 10, wherein the each respective plunger is engageable with the respective sealing device such that rotation of the plunger rotates the sealing device within the rotational path.
12. The mixing syringe assembly of any preceding clause, wherein the track is recessed within a wall of the respective barrel body.
13. The mixing syringe assembly of any preceding clause, wherein each sealing device comprises a projection which extends into the respective track.
14. The mixing syringe assembly of any preceding clause, wherein the sealing device comprises a one-way valve.
15. The mixing syringe assembly of any preceding clause, wherein the sealing device comprises a burstable film.
16. A method of assembling a mixing syringe assembly, the method comprising: inserting a first constituent material into a barrel body, wherein the barrel body comprises a track defining a first longitudinal path, a second longitudinal path, and a rotational path wherein the first longitudinal path and the second longitudinal path are rotationally offset from one another by the rotational path; and inserting a sealing device into the barrel body such that the barrel body is engaged within the rotational path and divides the barrel body into a first constituent material section and a second constituent material section, wherein the sealing device is rotatable within the rotational path such that rotation of the sealing device within the rotational path allows the sealing device to enter the second longitudinal path and be advanced along the second longitudinal path, and wherein the sealing device is operable to adjust from a sealing configuration to a non-sealing configuration to allow for the first constituent material from the first constituent material section to move into the second constituent material section holding a second constituent material.
17. The method of clause 16, wherein inserting the sealing device into the barrel body comprises engaging the sealing device to the first longitudinal path and advancing the sealing device along the first longitudinal path to the rotational path.
18. The method of any preceding clause, further comprising placing inserting the second constituent material into the second constituent material section.
19. The method of any preceding clause, further comprising inserting a plunger into the barrel body.
20. A method of using a mixing syringe assembly of any of clauses 1-15, the method comprising: advancing a plunger toward the sealing device to adjust the sealing device to the non-sealing configuration; further advancing the plunger toward the sealing device to transfer the first constituent material into the second constituent material section; engaging the plunger with the sealing device; twisting the plunger thereby causing the sealing device to rotate within the rotational path; and advancing the plunger and the sealing device along the second longitudinal path.

It should now be understood that the present disclosure relates to various mixing syringe assemblies and methods of mixing constituent materials with mixing syringe assemblies. The various embodiments provided herein may provide ready to use or easily assembled syringe assemblies for easily mixing components. Moreover, embodiments as provided herein may assist in maintaining sterility and/or constituent material integrity, while improving ease of mixing and delivery.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A mixing syringe assembly comprising:
a barrel body comprising a track defining a first longitudinal path, a second longitudinal path, and a rotational path wherein the first longitudinal path and the second longitudinal path are rotationally offset from one another by the rotational path; and
a sealing device configured to be positioned in a dividing position between within the rotational path, the sealing device dividing the barrel body into a first constituent material section and a second constituent material section, wherein the sealing device is rotatable within the rotational path such that rotation of the sealing device within the rotational path allows the sealing device to enter the second longitudinal path and be advanced along the second longitudinal path, and wherein the sealing device is operable to adjust from a sealing configuration to a non-sealing configuration to allow for a first constituent material from the first constituent material section to move into the second constituent material section holding a second constituent material.

2. The mixing syringe assembly of claim 1, further comprising: a plunger slidably positioned within the barrel body.

3. The mixing syringe assembly of claim 2, wherein the plunger is engageable with the sealing device such that rotation of the plunger rotates the sealing device within the rotational path.

4. The mixing syringe assembly of claim 1, wherein the track is recessed within a wall of the barrel body.

5. The mixing syringe assembly of claim 1, wherein the sealing device comprises a projection which extends into the track.

6. The mixing syringe assembly of claim 1, wherein the sealing device comprises a one-way valve.

7. The mixing syringe assembly of claim 1, wherein the sealing device comprises a burstable film.

8. A mixing syringe assembly for mixing multiple constituent components, the mixing syringe assembly comprising:
a first mixing syringe subassembly and a second mixing syringe subassembly coupled to the first mixing syringe subassembly, each mixing syringe subassembly comprising:
a barrel body comprising a track defining a first longitudinal path, a second longitudinal path, and a rotational path wherein the first longitudinal path and the second longitudinal path are rotationally offset from one another by the rotational path; and
a sealing device configured to be positioned within a dividing position between within the rotational path, the sealing device dividing the barrel body into a first constituent material section and a second constituent material section, wherein the sealing device is rotatable within the rotational path such that rotation of the sealing device within the rotational path allows the sealing device to enter the second longitudinal path and be advanced along the second longitudinal path, and wherein the sealing device is operable to adjust from a sealing configuration to a non-sealing configuration to allow for a first constituent material from the first constituent material section to move into the second constituent material section holding a second constituent material.

9. The mixing syringe assembly of claim 8, wherein the barrel body of the first mixing syringe subassembly is coupled to the barrel body of the second mixing syringe subassembly.

10. The mixing syringe assembly of claim 8, wherein the first mixing syringe subassembly and the second mixing syringe subassembly further comprise a plunger slidably positioned within the barrel body.

11. The mixing syringe assembly of claim 10, wherein the each respective plunger is engageable with the respective sealing device such that rotation of the plunger rotates the sealing device within the rotational path.

12. The mixing syringe assembly of claim 8, wherein the track is recessed within a wall of the respective barrel body.

13. The mixing syringe assembly of claim 8, wherein each sealing device comprises a projection which extends into the respective track.

14. The mixing syringe assembly of claim 8, wherein the sealing device comprises a one-way valve.

15. The mixing syringe assembly of claim 8, wherein the sealing device comprises a burstable film.

16. A method of assembling a mixing syringe assembly, the method comprising:
inserting a first constituent material into a barrel body, wherein the barrel body comprises a track defining a first longitudinal path, a second longitudinal path, and a rotational path wherein the first longitudinal path and the second longitudinal path are rotationally offset from one another by the rotational path; and
inserting a sealing device into the barrel body such that the barrel body is engaged within the rotational path and divides the barrel body into a first constituent material section and a second constituent material section, wherein the sealing device is rotatable within the rotational path such that rotation of the sealing device within the rotational path allows the sealing device to enter the second longitudinal path and be advanced along the second longitudinal path, and wherein the sealing device is operable to adjust from a sealing configuration to a non-sealing configuration to allow for the first constituent material from the first constituent material section to move into the second constituent material section holding a second constituent material.

17. The method of claim 16, wherein inserting the sealing device into the barrel body comprises engaging the sealing device to the first longitudinal path and advancing the sealing device along the first longitudinal path to the rotational path.

18. The method of claim 16, further comprising placing inserting the second constituent material into the second constituent material section.

19. The method of claim 16, further comprising inserting a plunger into the barrel body.

20. A method of using a mixing syringe assembly of claim 1, the method comprising:
advancing a plunger toward the sealing device to adjust the sealing device to the non-sealing configuration;
further advancing the plunger toward the sealing device to transfer the first constituent material into the second constituent material section;
engaging the plunger with the sealing device;
twisting the plunger thereby causing the sealing device to rotate within the rotational path; and
advancing the plunger and the sealing device along the second longitudinal path.
